(19) United States
(12) Patent Application Publication
(10) Pub. No.: US 2006/0059725 A1
(43) Pub. Date: Mar. 23, 2006

(54) Foot sole stimulation device for balance enhancement

(75) Inventors: Hen-Yu Lien, Taipei City (TW); Shu-Ling Yang, Taipei City (TW); Alice May-Kuen Wong, Taipei County (TW)

Correspondence Address: NIKOLAI & MERSEREAU, P.A., 900 SECOND AVENUE SOUTH, SUITE 820, MINNEAPOLIS, MN, 55402, US

(73) Assignee: CHANG GUNG UNIVERSITY, Tao-Yuan (TW)

(21) Appl. No.: 10/945,388

(22) Filed: Sep. 20, 2004

(51) Int. Cl. A61F 5/14

(52) U.S. Cl. 36/141

(57) ABSTRACT

A foot sole stimulation device includes a plate with stimulators on a top surface of the plate and arranged in a specific form. The stimulators are solid semi-circular balls with radius of 1.5 mm to 2.5 mm and arranged in a square matrix. A distance between the centers of adjacent rows and columns of the stimulators is 7 mm to 11 mm, and a distance between the closest peripheries of adjacent rows and columns of the stimulators is 4 mm to 6 mm. The device can be made as shoe soles, shoes or socks so as to provide gradual stimulation to the user who has problems of sense of balance. When users step on the stimulators, they compress and stimulate the skin to improve the ability of receptors in the sole of foot to detect change in different levels of mechanical stimulation. The device provides extra sensational information and information of the load allocations during standing, walking or turning so as to increase the ability of balance.

### FIELD OF THE INVENTION

The present invention relates to a foot sole stimulation device having stimulators arranged in a specific form to provide a gradual stimulation to the patient's foot sole so as to improve the balance function.

### BACKGROUND OF THE INVENTION

Human keep balance by gathering signals sensed by the peripheral nervous system and integrating these signals by the central nervous system. The information sensed by the nervous systems is used to judge the positioning in the space of human body, the movement of the center of mass of the body and the changes of the environment, and further adjust the tension of muscles of the body and limbs. Specific muscles are then activated to provide balance of the body and the limbs.

The skin of the foot sole is the only support surface for the human when walking and standing and the skin of the foot sole is the only part that contacts the floor. The sensation of the foot sole provides information about the surface conditions, the load allocations over the sole of foot and the sensation regarding the gravity relative to the supporting surface. The sensation relates to the behaviors of human body in dynamic and stationary balance. Accordingly, seniors and some patients suffered by stroke, cerebral paralysis may improve their balance by stimulating their foot sole. However, there is no proper disclosure about how much that the stimulation should be for the foot sole can help the seniors or patients to improve their balance.

Applicant spends a long period of time of search and development and tries to find a solution for providing a proper stimulation to the foot sole so as to improve the sense of balance for the users.

The present invention intends to provide a foot sole stimulation device which provides extra stimulation to the foot sole so as to increase the input of information about the balance and help users to improve their balance performance.

### SUMMARY OF THE INVENTION

The present invention relates to a stimulation device for providing extra stimulation to the foot sole so as to improve the balance performance for the seniors or patients who lack normal sense of balance.

The device includes a plate having stimulators which are solid semi-circular balls and each stimulator has a radius of 1.5 mm to 2.5 mm. The stimulators are arranged in a square matrix. A distance between two respective centers of adjacent rows and columns of the stimulators is 7 mm to 11 mm. A distance between closest peripheries of adjacent rows and columns of the stimulators is 4 mm to 6 mm.

When the user step on the stimulators, they compress and stimulate the skin to provide intensified sensation form the sole of foot which may improve the temporal and spatial resolution of the receptors reside in the sole of foot to mechanical stimulation. The device provides extra sensational information and information of the load allocations during standing, walking or turning so as to increase the ability of balance.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view of the stimulation device of the present invention;

FIG. 2 shows the top view and the side view of the stimulators on the device of the present invention;

FIG. 3 shows the device is made as a shoe sole,

FIG. 4 shows the comparison of results of using the device and results of without using the device.

### DETAILED DESCRIPTION OF THE PREFERRED EMBODIMENT

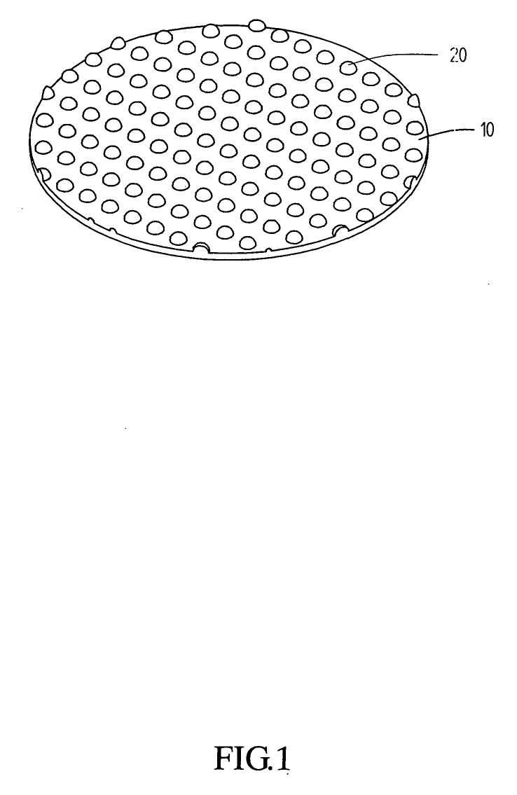

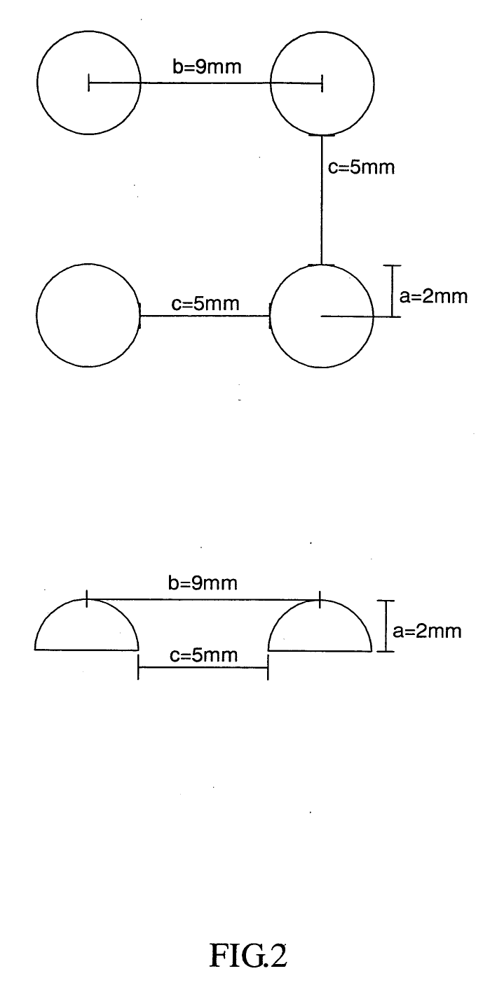

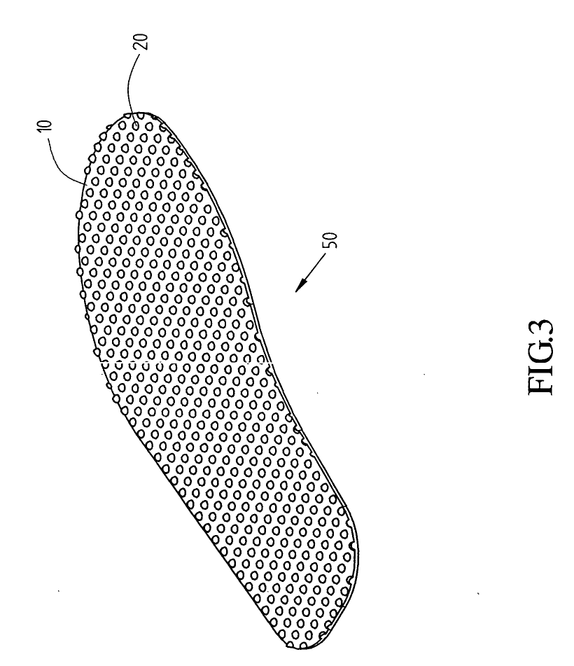

Referring to FIGS. 1 and 2, the foot sole stimulation device of the present invention comprises a plate 10 with stimulators 20 arranged in a specific form, wherein the plate 10 can be made as shoe soles 50 as shown in FIG. 3, shoes, and socks for stimulating the skin on the sole of feet.

The stimulators 20 are solid semi-circular balls with radius of 1.5 mm to 2.5 mm. In this embodiment, the radius is 2.0 mm. The stimulators 20 provide gradual stimulation to the users and are arranged in a square matrix. A distance between the centers of adjacent rows and columns of the stimulators 20 is 7 mm to 11 mm, in this embodiment, the distance is 9.0 mm. A distance between the closest peripheries of adjacent rows and columns of the stimulators 20 is 4 mm to 6 mm, in this embodiment, the distance is 5.0 mm. By the specific arrangement of the stimulators 20, the users receive a large amount of stimulation and will not over stimulate the users.

By using the device, extra stimulation can be obtained and the balance performance is improved.

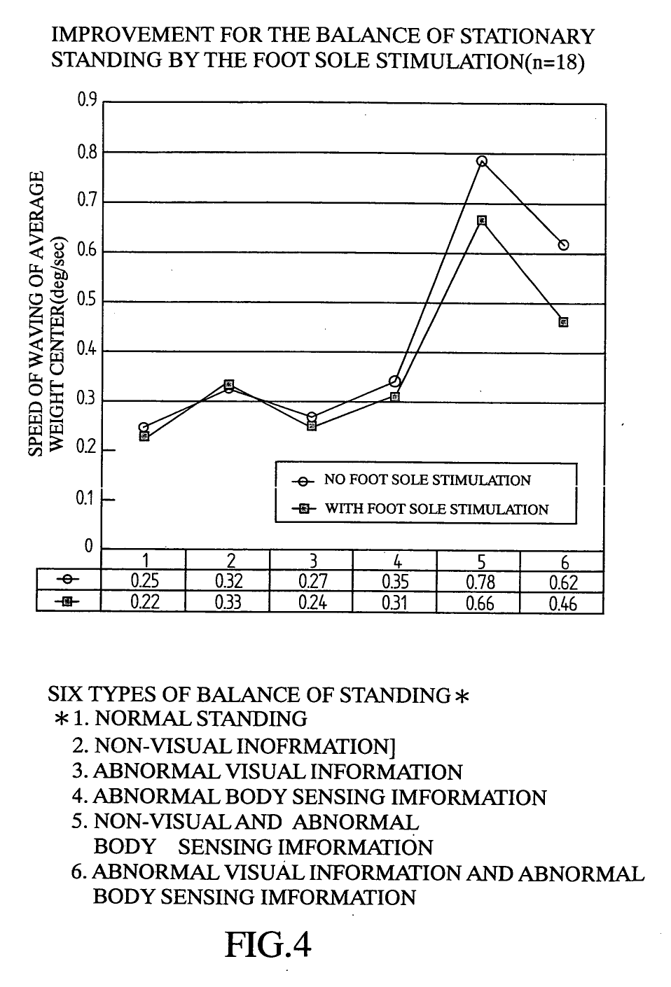

Referring to FIGS. 1, 3 and 4, the device can be made as a shoe sole 50 and when the user steps on the stimulators 20, the skin on the foot sole is stimulated gradually from the periphery to the center of the stimulators 20. In this embodiment, the skin is pressed up to 2 mm. The stimulation provides comfortable and proper intensity of stimulation which can be sensed by most mechanical receptors or sensors reside in the human skin. The area covered by the stimulators 20 with radius of 2 mm is smaller than the size of the receptive field of mechanical receptors in the sole of foot. The distance between centers of adjacent stimulators 20 is 9.0 mm which provides additional yet discriminating stimulation to the sole of foot. Applying this pattern of stimulation to the foot sole of the user provides enhanced sensation about load allocation over the contact surface and orientation of body relative to the foot sole. This enhanced sensation may further increases the capability of the skin receptors to detect changes in loading information of the foot sole during movement such as standing, walking or turning so as to increase the ability of balance.

The function of the device for the balance of standing is proved by the Smart Balance Master System (Neurocom International Inc., Oregon, USA). After being stimulated by the device of the present invention, under the circumstances of interruption in visual and body senses which seniors usually encounter, the speed and altitude of waving of body is obviously reduced and the user has better balance of standing.

Those seniors and patients who have abnormal balance sense such as those suffered by stroke and cerebral paralysis may improve their sense of balance after they used the shoe soles, socks or therapeutic pads which adapted from this device. The device provides extra sensational information and information of the load allocations during standing, walking or turning so as to obtain better balance performance.

While we have shown and described the embodiment in accordance with the present invention, it should be clear to those skilled in the art that further embodiments may be made without departing from the scope of the present invention.

What is claimed is:

1. A stimulation device comprising:
a plate having stimulators which are solid semi-circular balls and each stimulator having a radius of 1.5 mm to 2.5 mm, the stimulators arranged in a square matrix, a distance between two respective centers of adjacent rows and columns of the stimulators being 7 mm to 11 mm, a distance between closest peripheries of adjacent rows and columns of the stimulators being 4 mm to 6 mm.

2. The device as claimed in claim 1, wherein the radius of each stimulator is 2.0 mm, the distance between the two respective centers of stimulators is 9.0 mm and the distance between the closest peripheries of adjacent rows of the stimulators is 5.0 mm.
